(19) United States
(12) Patent Application Publication
(10) Pub. No.: US 2022/0047739 A1
(43) Pub. Date: Feb. 17, 2022

(54) Modular ultraviolet light panel

(71) Applicant: Nemesis UVC LLC, Garland, TX (US)

(72) Inventors: Eric L. Wade, Garland, TX (US); John Early, Garland, TX (US); Aaron Wade, Garland, TX (US)

(21) Appl. No.: 17/401,003

(22) Filed: Aug. 12, 2021

Related U.S. Application Data
(60) Provisional application No. 63/064,740, filed on Aug. 12, 2020.

(51) Int. Cl. A61L 2/10; A61L 9/22; A61L 2/14

(52) U.S. Cl. CPC A61L 2/10; A61L 9/22; A61L 2/14; A61L 2209/14; A61L 2202/14; A61L 2209/12; A61L 2209/111; A61L 2202/11

(57) ABSTRACT

A UV light unit. The UV light unit may comprise one or more components which 1) expose surfaces, areas, and spaces to UV light sufficient to disinfect and/or sanitize the surfaces, areas, and spaces, 2) provide one or more non-UV light sources configured to illuminate the surfaces, areas, and spaces, and 3) provide air filtration/purification or cleaning.

### CROSS REFERENCE TO RELATED APPLICATIONS

In accordance with 37 C.F.R. 1.76, a claim of priority is included in an Application Data Sheet filed concurrently herewith. Accordingly, the present invention claims priority to U.S. Provisional Patent Application No. 63/064,740, entitled “MODULAR ULTRAVIOLET LIGHT PANEL”, filed Aug. 12, 2020. The contents of the above referenced application are incorporated herein by reference in their entirety.

### FIELD OF THE INVENTION

The present invention relates to devices, systems, and methods for lighting and/or disinfecting an area; to devices and methods for housing one or more ultraviolet (UV) lights or panels to prevent microbial or pathogen growth within an area; and more particularly, to a modular ultraviolet (UV) light unit for use in preventing microbial or pathogen growth within an area.

### BACKGROUND OF THE INVENTION

The need for preventing or minimizing microbial growth is more important than ever before. As demonstrated by the 2020 recent outbreak of COVID 19, and despite modern medicine that is more advanced than at any time in our history, humans remain susceptible to deadly viral outbreaks. While the world is racing to find novel cures or vaccines, the need to develop strategies to minimize exposure to the disease is just as important. Even if we are able to stop this disease soon, the next deadly outbreak may be just around the corner. Finding ways to safely live and function with COVID 19, or any other deadly viruses, is critical to world stability.

Accordingly, strategies for maintaining surfaces and spaces clean and microbial free are critical. New devices that can be used to sanitize or disinfect rooms, areas, or other high use spaces are needed.

### SUMMARY OF THE INVENTION

The present invention describes devices and methods for disinfecting an area using ultraviolet (UV) lights. The devices and methods include a fixed or movable UV light unit. The UV light unit includes one or more of the following functions: 1) expose surfaces, areas, and spaces to UV light sufficient to disinfect and/or sanitize the surfaces, areas, and spaces, 2) provide one or more non-UV light sources configured to illuminate the surfaces, areas, and spaces, and 3) provide air filtration or cleaning by exposure of the air associated with the surfaces, areas, and spaces to the UV light. The UV light device may also be configured to cycle between clean mode and visible light mode.

Accordingly, it is an objective of the invention to provide a unique disinfecting and/or sanitizing device and method.

It is a further objective of the invention to provide disinfecting and/or sanitizing devices and methods using UV light.

It is a still further objective of the invention to provide a UV light based device configured to disinfect and/or sanitize surfaces or areas.

It is a further objective of the invention to provide a modular UV light unit configured to disinfect and/or sanitize large surfaces or areas.

It is a further objective of the invention to provide disinfecting and/or sanitizing devices and methods using UV light and non-UV light.

It is yet another objective of the instant invention to provide disinfecting and/or sanitizing devices, using UV lights, which are also configured to provide air filtration/purification.

It is yet another objective of the instant invention to provide a light unit which disinfects and/or sanitizes surfaces or areas using UV light, illuminates the same surfaces or areas using non-UV light, such as LEDs, and can clean or filter or purify air associated with the same surfaces or areas.

It is a still further objective of the invention to provide a light unit which is configured to automatically disinfect and/or sanitize surfaces or areas using UV light when safe to do so.

It is a still further objective of the invention to provide a UV light pod configured to disinfect and/or sanitize large surfaces or areas in combination with one or more non-UV light sources configured to illuminate a space, surface, or area.

It is a still further objective of the invention to provide a UV light pod configured to disinfect and/or sanitize an area or surface which uses an in-use warning system to prevent unwanted UV light exposure.

It is yet another objective of the invention to provide a UV light pod configured to disinfect and/or sanitize an area or surface which uses an occupant detection sensor system to prevent unwanted UV light exposure.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

### BRIEF DESCRIPTION OF THE FIGURES

FIG. 1 is a perspective view of an illustrative embodiment of a UV light unit;

FIG. 2 is an alternative perspective view of the UV light unit illustrated in FIG. 1;

FIG. 3 is a bottom view of the UV light unit illustrated in FIG. 1;

FIG. 4 is a back view of the UV light unit illustrated in FIG. 1;

FIG. 5 is a side view of the UV light unit illustrated in FIG. 1;

FIG. 6 is an exploded view of the UV light unit illustrated in FIG. 1;

FIG. 7 is an alternative bottom view of the UV light unit illustrated in FIG. 1;

FIG. 8 is a bottom view of the UV light unit illustrated in FIG. 1, shown with the LED light lens and sanitizing/disinfecting (UV) light source face plate removed;

FIG. 9 is a bottom view of the UV light unit illustrated in FIG. 8, shown with the sensor panel and the safety feature panel removed;

FIG. 10 is a perspective view of the UV light unit with air filtration;

FIG. 11 is an alternative perspective view of the UV light unit with air filtration illustrated in FIG. 10;

FIG. 12 is a side view of the UV light unit with air filtration illustrated in FIG. 10;

FIG. 13 is a front view of the UV light unit with air filtration illustrated in FIG. 10;

FIG. 14 is a bottom view of the UV light unit with air filtration illustrated in FIG. 10;

FIG. 15 is an exploded view of the UV light unit with air filtration illustrated in FIG. 10;

FIG. 16 is an alternative view of the exploded view of the UV light unit with air filtration illustrated in FIG. 10;

FIG. 17A illustrates the upper frame and the air filtration enclosure unit of the UV light unit with air filtration illustrated in FIG. 10;

FIG. 17B is an alternative view of the UV light unit with air filtration illustrated in FIG. 10;

FIG. 18 illustrates the lower frame associated with the UV light unit with air filtration illustrated in FIG. 10;

FIG. 19A illustrates the air filtration enclosure unit of the UV light unit with air filtration illustrated in FIG. 10;

FIG. 19B is an alternative view of the air filtration enclosure unit shown in FIG. 19A;

FIG. 19C is a side view of the air filtration enclosure unit shown in FIG. 19B;

FIG. 19D is an illustrative example of a connector plate;

FIG. 20A illustrates a portion of the air filtration enclosure unit with air intake vents;

FIG. 20B illustrates a portion of the air filtration enclosure unit with air outtake vents;

FIG. 21A is a perspective view of an alternative embodiment of the UV light unit;

FIG. 21B is a corresponding view of the UV light unit illustrated in FIG. 21A;

FIG. 22A is a bottom view of the UV light unit illustrated in FIG. 21A;

FIG. 22B is a corresponding view of the UV light unit illustrated in FIG. 22A;

FIG. 23A is a front view of the UV light unit illustrated in FIG. 21A;

FIG. 23B is a corresponding view of the UV light unit illustrated in FIG. 23A;

FIG. 24A is a side view of the UV light unit illustrated in FIG. 21A;

FIG. 24B is a corresponding view of the UV light unit illustrated in FIG. 24A;

FIG. 25 is an exploded view of the UV light unit illustrated in FIG. 10A;

FIG. 26 is an illustrative embodiment of a power connector with bracket;

FIG. 27 is a perspective view of an alternative embodiment of a housing unit of the UV light unit;

FIG. 28 is an exploded view of the housing unit of the UV light unit illustrated in FIG. 27;

FIG. 29 is an exploded view of the housing unit of the UV light unit illustrated in FIG. 27;

FIG. 30 is an exploded view of an alternative embodiment of a housing unit of the UV light unit;

FIG. 31 is an exploded view of an alternative embodiment of a housing unit of the UV light unit; and

FIG. 32 is an exploded view of an alternative embodiment of a housing unit of the UV light unit.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

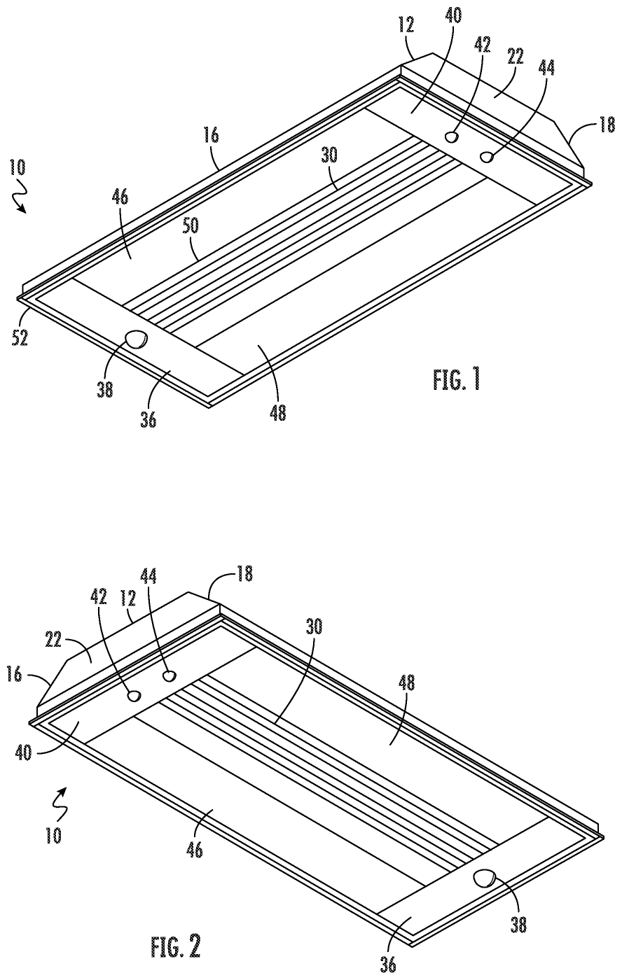

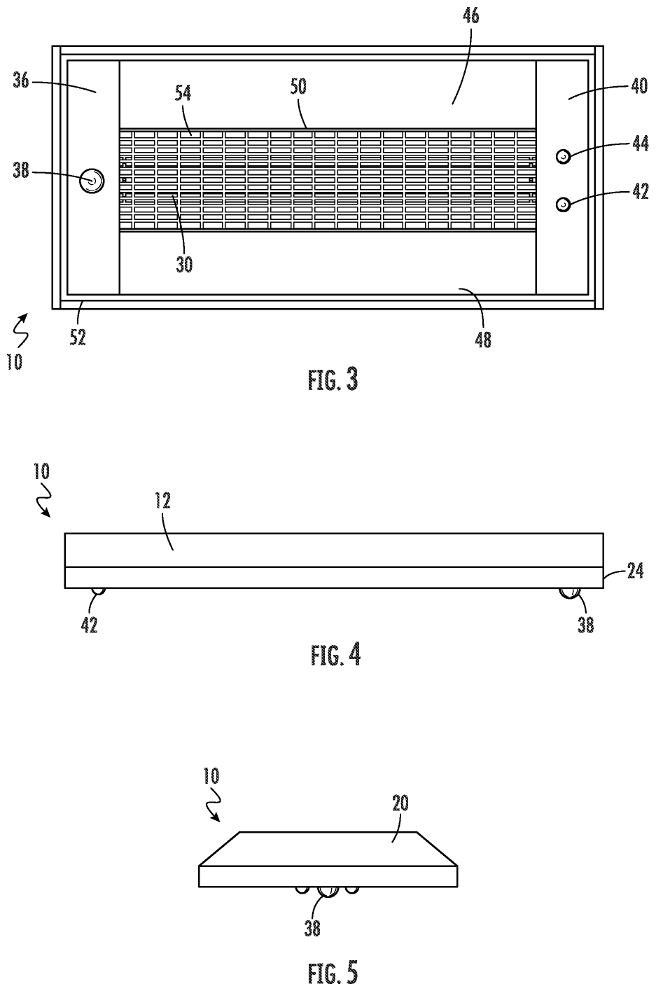

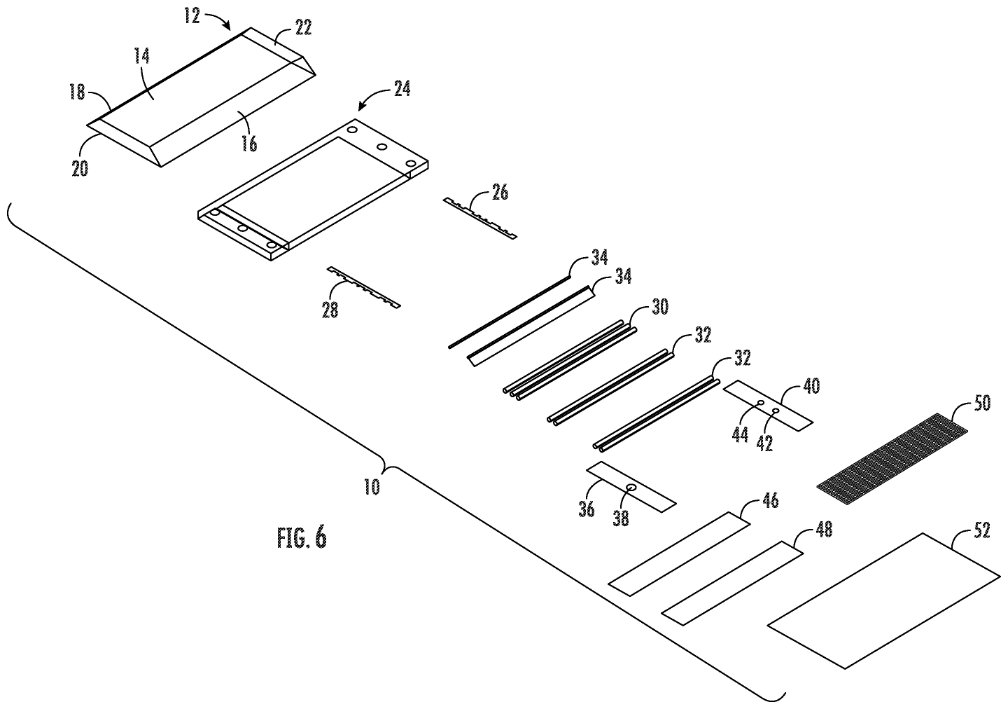

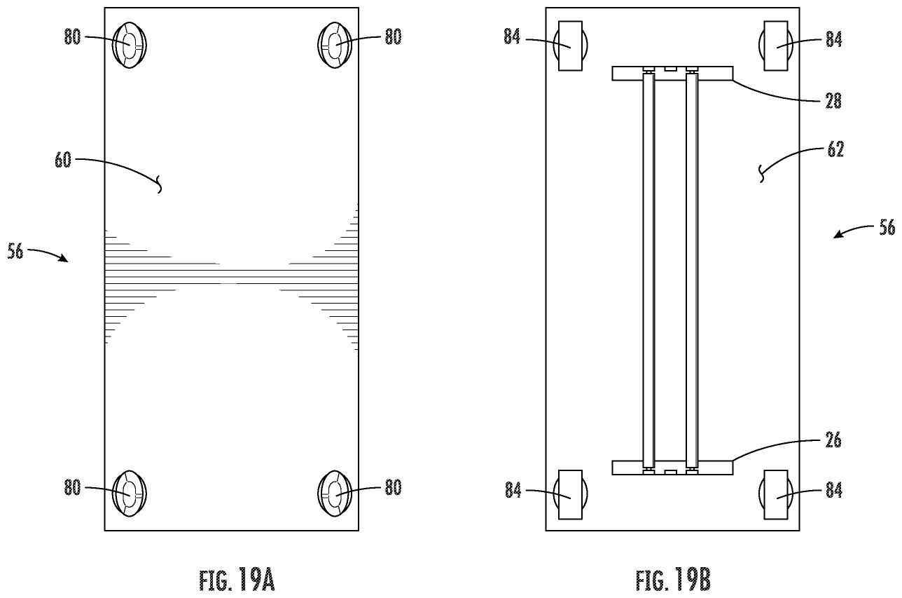

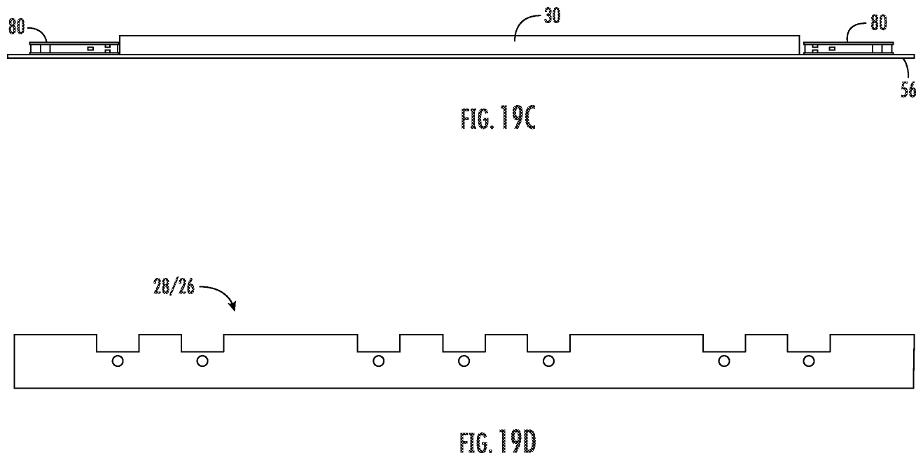

Referring to FIGS. 1-6, an illustrative embodiment of an ultraviolet (UV) light based sanitization device, referred to generally as a UV light unit 10, is shown. The UV light unit is configured to expose surfaces, areas, and spaces to UV light sufficient to disinfect and/or sanitize the surfaces, areas, and spaces, 2) provide one or more non-UV light sources configured to illuminate the surfaces, areas, and spaces, and 3) provide air filtration/purification or cleaning. The UV light unit 10 may be made of any suitable material, such as stainless steel, or combinations of suitable materials. The UV light unit may comprise an upper frame 12 having a generally planar upper surface or top 14, two opposing sides 16 and 18, a left end surface 20 and a right end surface 22. A lower frame 24 secures to the upper frame 12 via connector plates 26 and 28 (see also FIG. 19D) with the use of mechanical fasteners, such as small screws (not shown). Once secured together, the upper frame 12 and the lower frame 24 provide an enclosed area which may be used to house the UV light units 10 functional components, such as a control unit, circuit board, ballast (triple ballast), driver, electrical wiring, built in timer, or WIFI units, processors, memory, electrical outlet receptacles, power cord receptacles, timer units, fuses, terminal blocks, transformers, or WIFI switches (not shown), wireless technology components such WIFI/RF/BLUETOOTH transmitters and/or receivers). One or more of the functional components may be attached to the end plate and within those compartments.

The UV light unit 10 is preferably configured to provide both sanitizing/disinfecting light and illuminating light. Secured to the lower frame 24 are one or more sanitizing/disinfecting light sources 30, such as a standard UV light lamp or an LED UV lamp. Preferably, one or more sanitizing/disinfecting light sources 30 are LED UVC bulbs (emitting in the range of 200-400 nm, preferably in the range of approximately 265 nm to about 280 nm). The UV light unit 10 may be configured so that when powered ON (OFF), all of the one or more sanitizing/disinfecting light sources 30 turn ON (OFF) and emit light at the same time, or each individual sanitizing/disinfecting light source 30 may be turned ON (OFF) and emit light separately and independently. The UV light unit 10 may also include other non-UV light producing lamps 32, such as LED lights, RGB LED 24/7 house or work lights for use in typical day to day illumination of the area or surface. The UV light unit 10 may include one or more reflectors to direct the UV light or non-UV light directionally. The UV light unit 10 may also include one or more mounting plates, 34, for mounting any of the safety equipment.

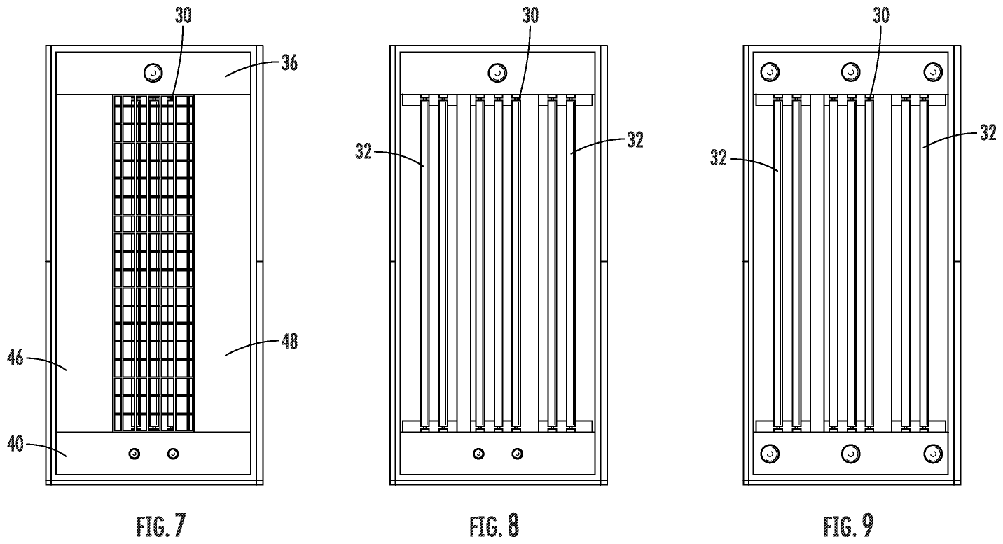

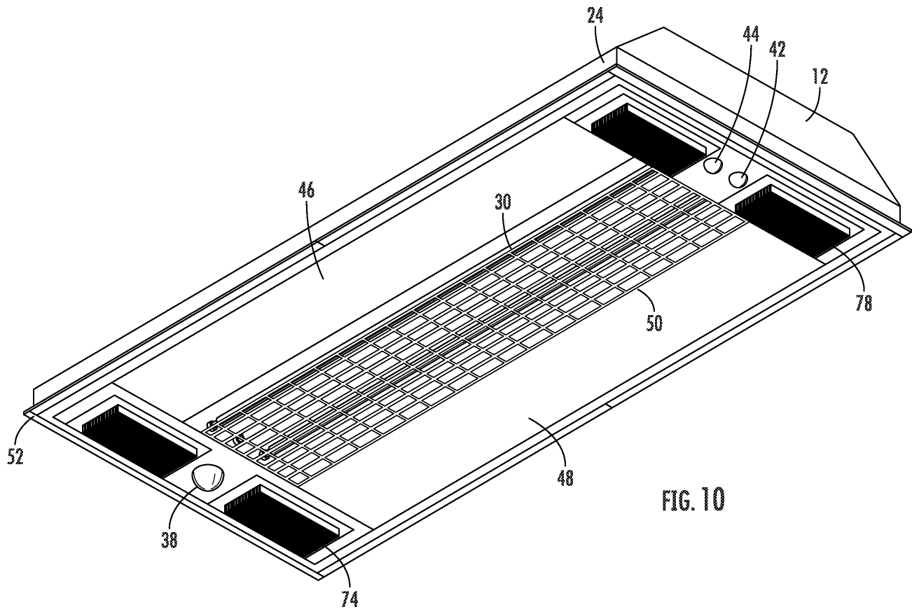

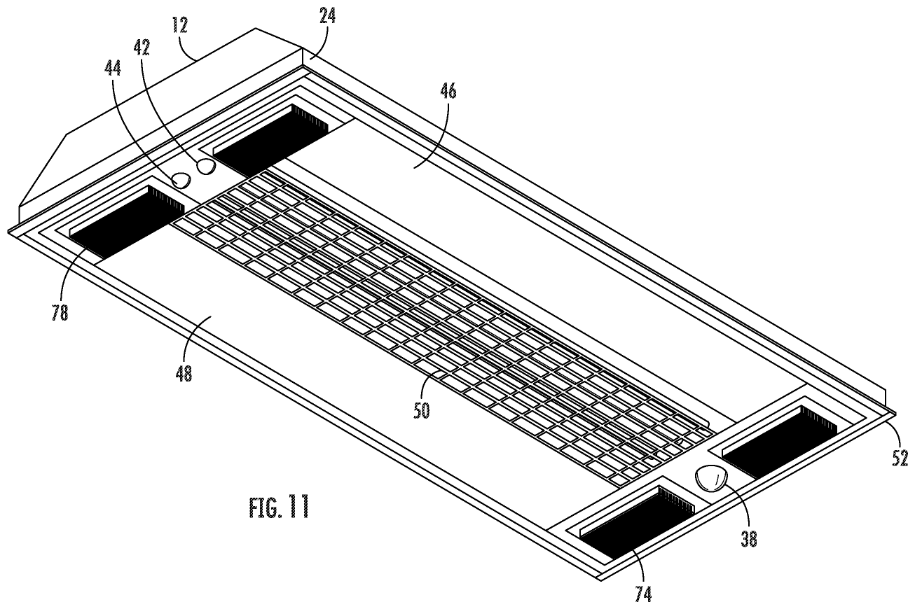

The non-UV light producing lamps 32 may be positioned and/or secured to the lower frame 24 in a location separate from the one or more sanitizing/disinfecting light sources 30. As illustrated in FIGS. 7-9, non-UV light producing lamps 32, i.e. LED lights, are oriented to the left and the right of the one or more sanitizing/disinfecting light sources 30, i.e. LED UVC bulbs. With the capability to expose surfaces, areas, rooms, or spaces to UV light and non-UV light, the UV light unit comprises dual functionality. To effectively provide dual functionality, the UV light unit 10 may be configured to first expose the surfaces, areas, rooms, or spaces to visible light. When the surfaces, areas, rooms, or spaces no longer require use of visible light, or to disinfect and/or sanitize the surfaces, areas, rooms, or spaces, the UV light unit 10 may be designed to automatically switch to producing UV light to allow the surfaces, areas, rooms, or spaces to be exposed to such light. Once the predetermined amount of time the surfaces, areas, rooms, or spaces have been exposed to the UV light passes, or if a user requires the use of visible light, the UV light production is terminated.

To provide such functionality, the UV light unit 10 may contain one or more safety or operational control components. A sensor panel 36 with one or more occupancy sensor, such as a motion sensor 38 (or alternatively, heat/thermal sensor) may be secured to the lower frame 24. The motion sensor 38, which may be a microwave motion sensor, is designed to detect motion, such as a person moving about within the space, area, or room in which the UV light unit 10 is placed therein and being exposed to UV light (disinfection range). If motion is detected, the UV light unit 10 is programmed to allow one or more of the non-UV light producing lamps 32 to be turned on (and terminate the UV light emission or exposure), thus exposing the surfaces, space, area, or room to visible light. After a predetermined time period of no motion sensed, the UV light unit 10 may be programmed to deactivate, i.e. turn off, the one or more non-UV light producing lamps 32. Upon deactivation, the UV light unit 10 would be programmed to then activate, or turn on, the one or more sanitizing/disinfecting light sources 30. This action exposes the surfaces, space, area, or room to UV light. Accordingly, the one or more occupancy sensors are operatively configured to immediately shut off the UV light unit 10 (i.e. stop the UV lights 30 from operating) if a person or animal is within the disinfection range or opens a door while the UV light unit 10 is in the “ON” or operating position, thus preventing accidental human/animal UV light exposure when the UV lights are operating.

In a preferred embodiment, the UV light unit 10 would be programmed to switch to a clean mode, allowing the UV light to be turned ON for a predetermined run time period to allow for complete disinfection or sanitization. After the time period ends, the one or more sanitizing/disinfecting light sources 30 are programmed to deactivate, or turn OFF. If the motion sensor 38 detects motion while the one or more sanitizing/disinfecting light sources 30 are in the ON position as part of the auto run period, the one or more sanitizing/disinfecting light sources 30 would automatically shut OFF, and the one or more non-UV light producing lamps 32 would automatically turn ON.

To prevent exposing a person to UV light when the UV light unit 10 is in the clean mode and exposing surfaces, spaces, areas, or rooms to UV light, the UV light unit 10 may utilize one or more safety features. For example, a safety feature panel 40 may be secured to the lower frame 24. The safety feature panel 40 may include a non-UV light indicator 42, illustrated herein as a visual red LED light indicator, and an audible alarm device 44 (yellow or green light) (with speaker) to alert the user when the UV light is in the ON operation.

The UV light unit 10 may include LED diffusion lenses 46, 48 for covering the one or more non-UV light producing lamps 32. The UV light unit 10 may also include a hinge front cover that may be configured to swing down for full access to components and lamps (i.e. one or more sanitizing/disinfecting light sources 30) for ease of servicing, and a sanitizing/disinfecting (or UV) light source face plate 52, sized and shaped to cover the one or more sanitizing/disinfecting light sources 30. The sanitizing/disinfecting (or UV) light source face plate 52 preferably has openings or gratings 54 to allow UV light exposure.

The UV light unit 10 may also be used to provide air filtration/purification functionality. Accordingly, the UV light unit 10 may be configured to drive air into the area between the upper frame 12 and the lower frame 24 and to one or more UV lights. The air is cleaned and purified as it is drawn over the one or more sanitizing/disinfecting light sources 30 prior to exiting the area. As such, the UV light unit 10 may be configured to allow the interior room air (i.e. the room where the UV light unit 10 is placed) to be pulled through HEPA filter intakes on a single end of the UV light unit 10 with high power fans that force the air into an upper UVC chamber to cause the air to flow over the one or more sanitizing/disinfecting light sources 30, preferably high output UVC, sanitizing the air once inside and expelling it through more HEPA filters to recirculate into the room. The fans are high power enough to recirculate the interior room air over six times per hour.

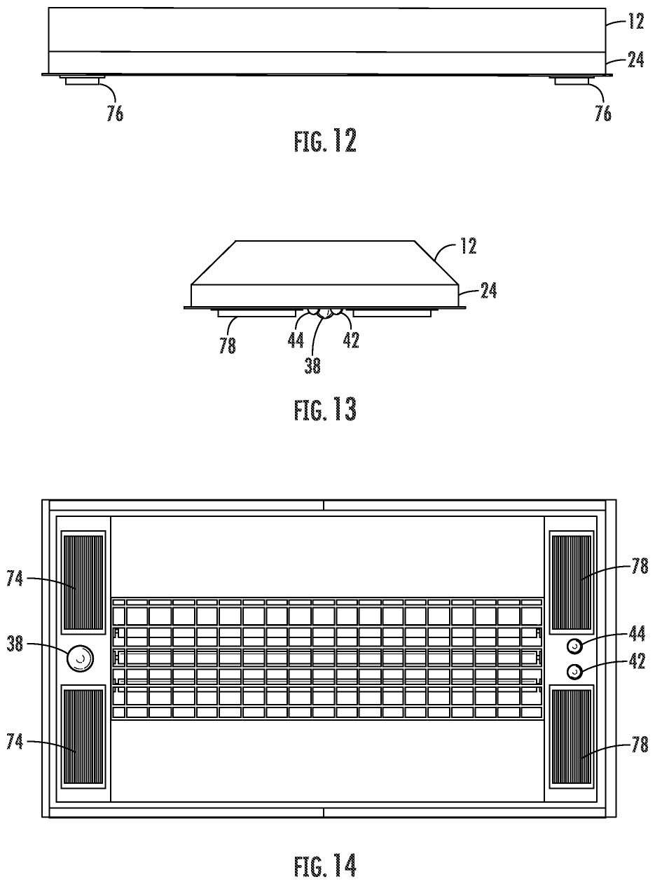

Referring to FIGS. 10-16, the UV light unit 10 shown is configured with air filtration. The UV light unit 10 in this embodiment includes one or more, or all of the same features or components as described above, with the inclusion of air filtration features. Secured to the upper frame 12 and/or the lower frame 24 is an air filtration enclosure unit 56 having an elongated, generally planar plate or main body 58 having a first surface 60 and a second opposing surface 62. Together, the upper frame 12, the lower frame 24, and the air filtration enclosure unit 56 may be referred to as the air purification module. The air filtration enclosure unit 56 rests or is secured between the upper frame 12 and/or the lower frame 24, enclosing the interior 64 of the upper frame 12.

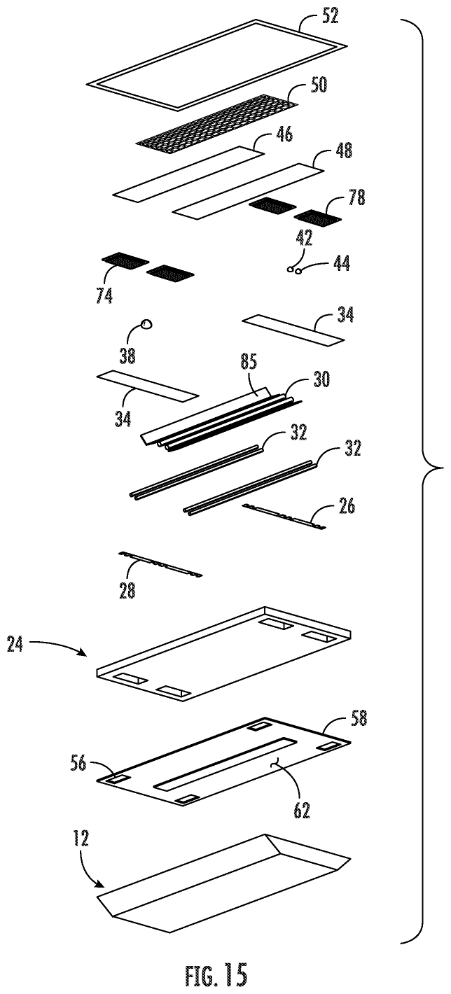

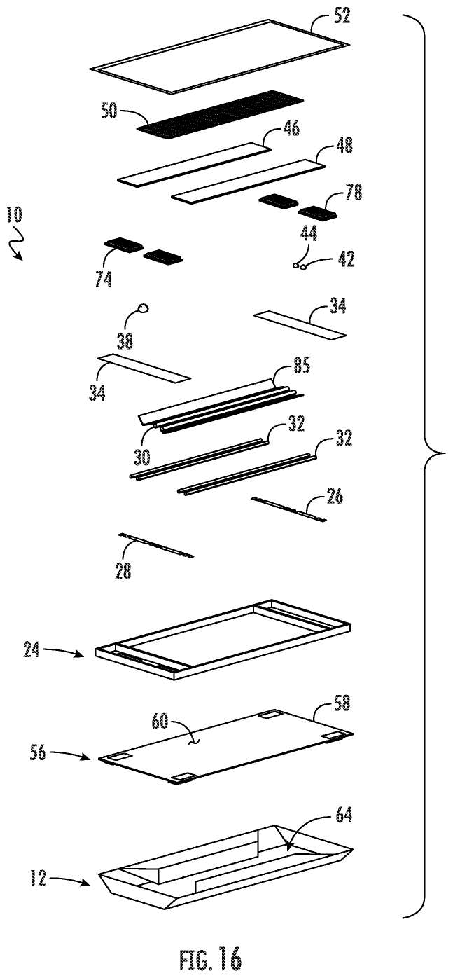

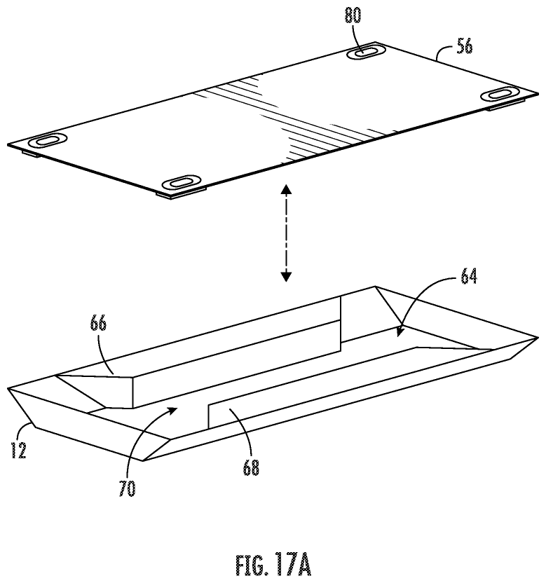

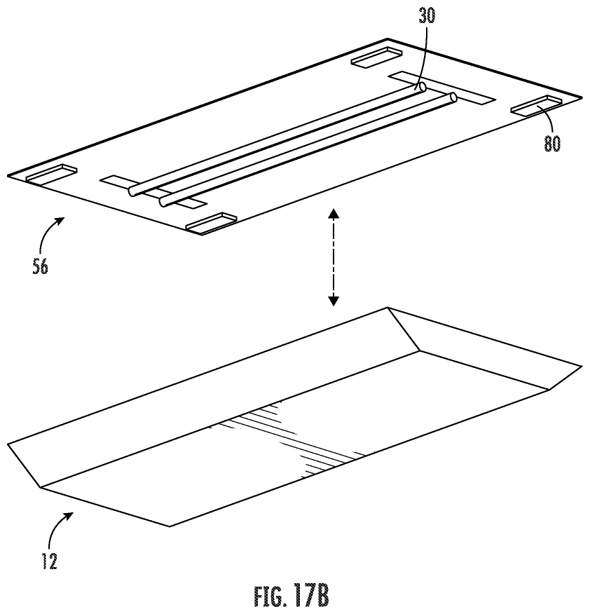

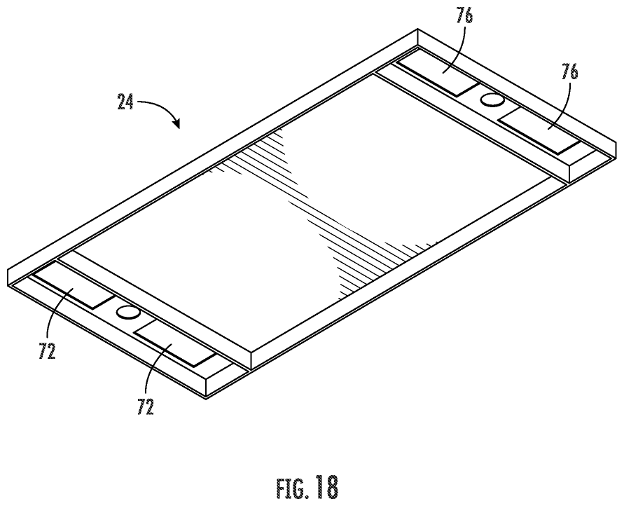

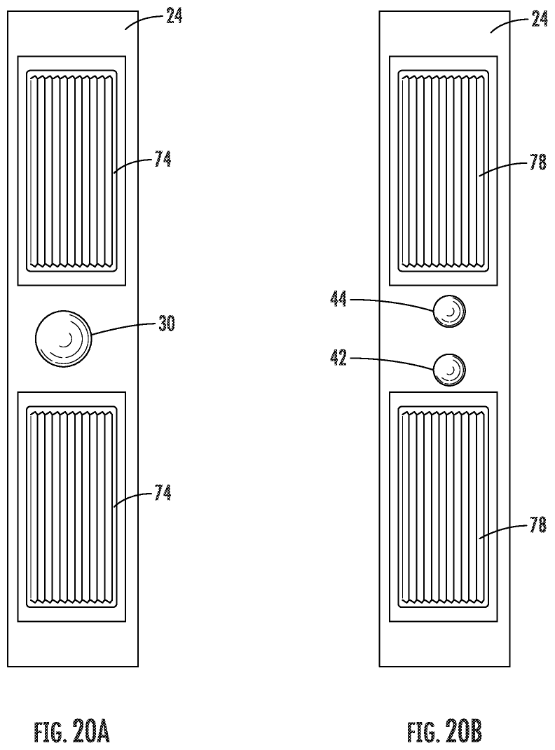

The interior 64 of the upper frame 12 may include opposing ledges or ridges 66 and 68, thus creating an air channel 70, see FIG. 17A. The lower frame 24 contains cut-out sections 72 sized and shaped to receive intake vents 74, and cut-out sections 76 sized and shaped to receive outtake vents 78, see FIG. 18, FIG. 20A and FIG. 20B. Preferably, the intake vents 74 and/or the outtake vents 78 include a filter, such as a HEPA filter. The air filtration enclosure plate 58 may include one or more fans 80, secured to the thereto via fan support plates 84, see FIGS. 19A, 19B and 19C. The air filtration enclosure plate second opposing surface 62 is shown with the one or more sanitizing/disinfecting light sources 30 secured thereto via connector plates 26 and 28. The one or more sanitizing/disinfecting light may also be partially enclosed by a reflector plate 85, see FIGS. 15 and 16, made of mirrored metal.

The UV light unit 10 may include a WIFI switch with power cord (not shown), such as a SONOFF WIFI connector, to control one or more of the above described functionalities, such as turn ON/OFF the one or more sanitizing/disinfecting light sources 30 or the one or more non-UV light producing lamps 32 via an app. The clean mode/sanitize mode (such as how long the sanitizing/disinfecting light sources 30 are turned on) may also be controlled by an app. The UV light unit 10 may be configured with radiofrequency (RF) control or operation. The UV light unit 10 may utilize other wireless technology, such as BLUETOOTH.

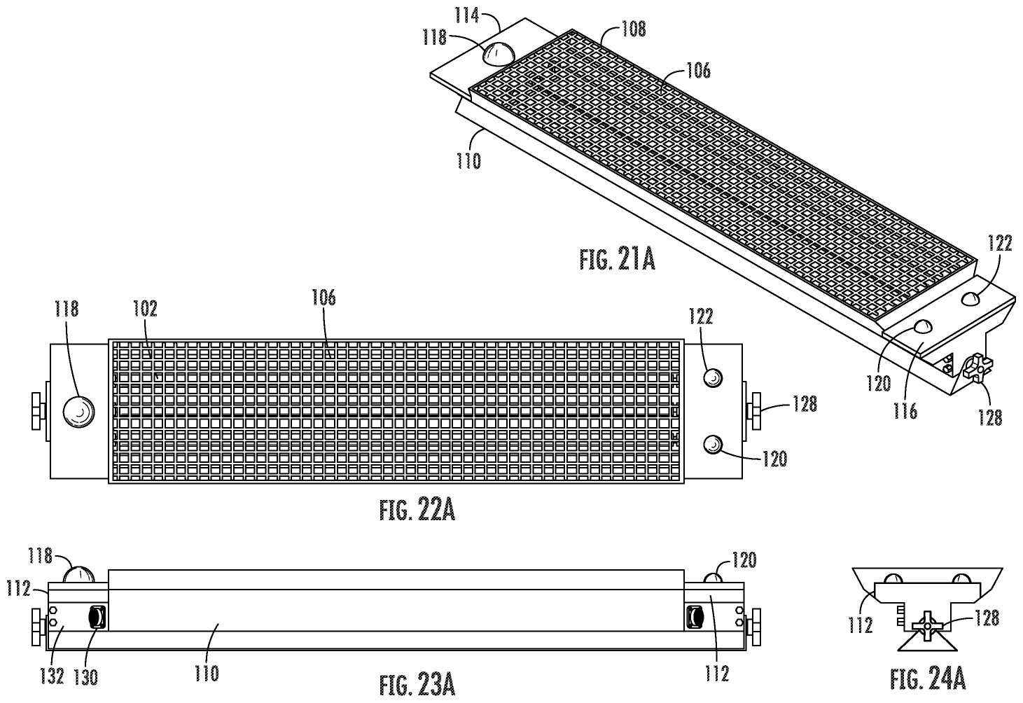

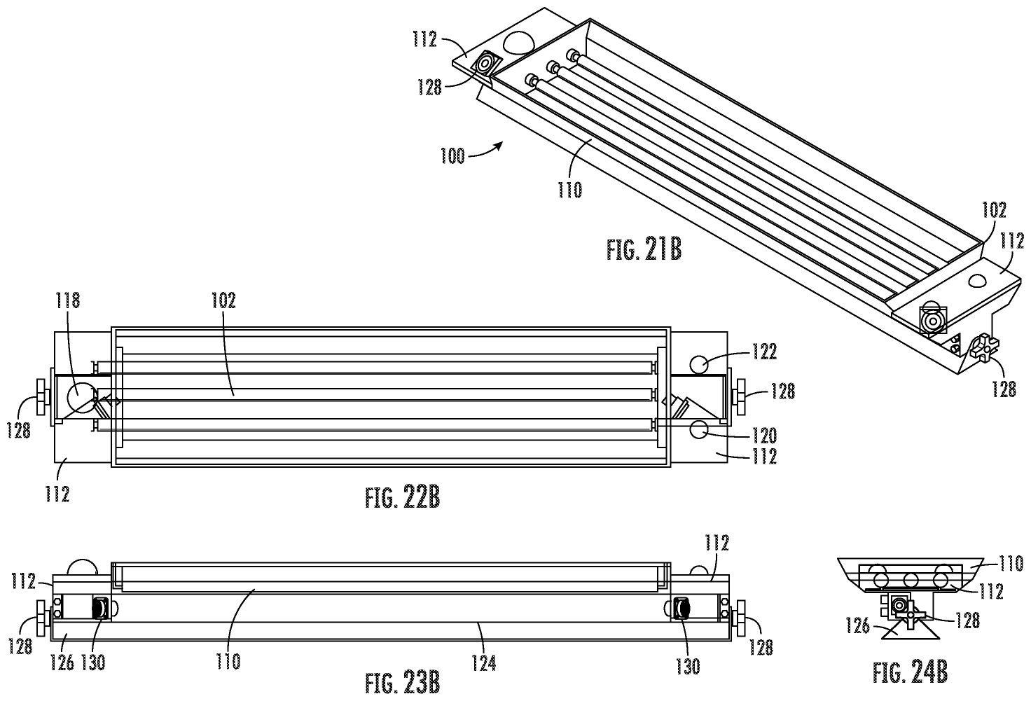

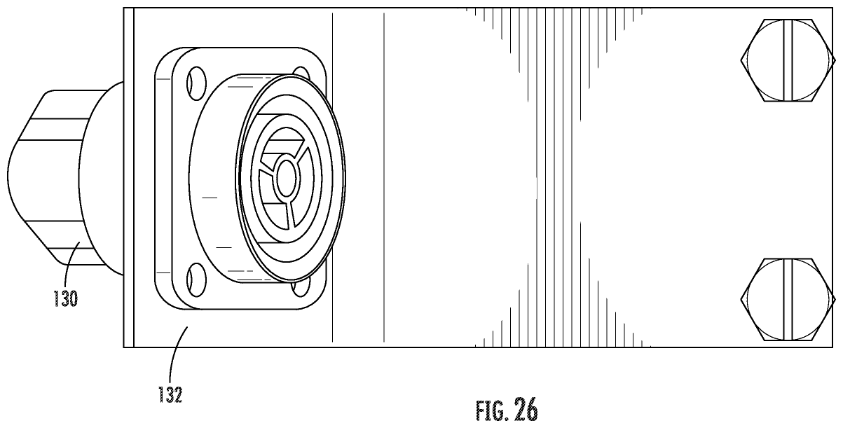

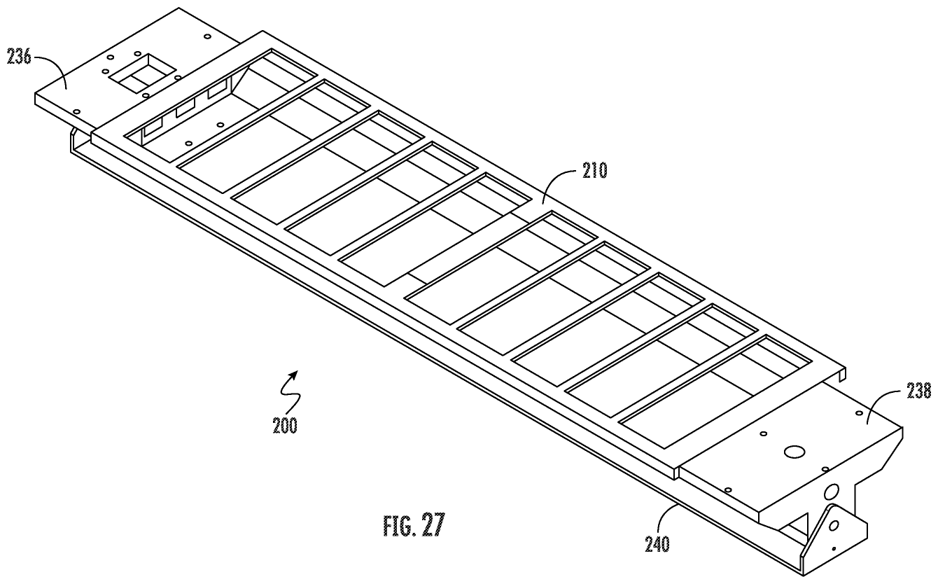

Referring to FIGS. 21A-26, an alternative embodiment of the UV light unit, referred to generally as UV light unit 100, is shown. The UV light unit 100 has many of the same features as that described for the UV light unit 10. The UV light unit 100 is configured to expose surfaces, areas, and spaces to UV light sufficient to disinfect and/or sanitize the surfaces, areas, and spaces, and 2) provide air filtration/purification or cleaning. The UV light unit 100 may be made of any suitable material, such as stainless steel, or combinations of suitable materials. The UV light unit 100 may include one or more sanitizing/disinfecting light sources 102, such as a standard UV light lamp or an LED UV lamp. Preferably, the one or more sanitizing/disinfecting light sources 102 are LED UVC bulbs (emitting in the range of 200-400 nm, preferably in the range of approximately 265 nm to about 280 nm). The UV light unit 100 may be configured so that when powered ON (OFF), all of the one or more sanitizing/disinfecting light sources 102 turn ON (OFF) and emit light at the same time, or each individual sanitizing/disinfecting light source 102 may be turned ON (OFF) and emit light separately and independently. As an illustrative example, the UV light unit 100 (and the UV light unit 10) may include three UV-C output lights at least capable of surface disinfection of up to 15′×15′ with 10′ ceilings.

The one or more sanitizing/disinfecting light sources 102 may be placed between a reflector 104 and a sanitizing/disinfecting (or UV) light source face plate 106. The sanitizing/disinfecting (or UV) light source face plate 106 preferably has openings or gratings 108 to allow UV light exposure. The one or more sanitizing/disinfecting light sources 102 and reflector 104 may be housed within an interior 109 of a back plate 110. The back plate 110 may be made of or have a reflective material. The back plate 110 may have off set or flanged portions 112 sized and shaped to receive and secure therein a sensor panel 114 and a safety feature panel 116. The sensor panel 114 includes one or more occupancy sensor(s), such as a motion sensor 118, (or alternatively, heat/thermal sensors, which may be a microwave motion sensor designed to detect motion, such as a person moving about within the space, area, or room in which the UV light unit 100 is placed therein.

If motion is detected, the UV light unit 100 is programmed to turn OFF the one or more sanitizing/disinfecting light sources 102. After a predetermined time period of no motion sensed, the UV light unit 100 may be programmed to activate, i.e. turn ON, one or more sanitizing/disinfecting light sources 102. This action exposes the surfaces, space, area, or room to UV light. The UV light unit 100 may be programmed to be switched to a clean mode, allowing the UV light to be turned on for a predetermined run time period to allow for complete disinfection or sanitization when the motion sensor 118 does not detect motion for a predetermined time, such as for 30 seconds or 1 minute. After the time period ends, the one or more sanitizing/disinfecting light sources 102 are programmed to deactivate, or turn OFF. If the motion sensor 118 detects motion while the one or more sanitizing/disinfecting light sources 102 are in the ON position, the one or more sanitizing/disinfecting light sources 102 would be shut OFF. Accordingly, the one or more occupancy sensors 118 are operatively configured to immediately shut off the UV light unit 100 (i.e. stop the one or more sanitizing/disinfecting light sources 102 from operating) if a person or animal is within the disinfection range or opens a door while the UV light unit 100 is in the “ON” or operating position, thus preventing accidental human/animal UV light exposure when the UV lights are operating.

To prevent exposing a person to UV light exposure when the UV light unit 100 is operating in the clean mode and exposing surfaces, spaces, areas, or rooms to UV light, the UV light unit 100 may utilize one or more safety features. For example, the safety feature panel 116 may include a non-UV light indicator 120, illustrated herein as a visual red LED light indicator, and an audible alarm 122 (and speaker) to alert the user when the UV light is in the ON operation.

A lower cover 124 secures to the back plate 112 to enclose the back plate 112. One or more functional components that drive the UV light unit 100 function or control a function may be stored within the back plate 112, such as a control unit, processing unit, circuit board, ballast (triple ballast), driver, electrical wiring, power cords, built in timer, or WIFI units, control panels or circuit boards, processors, memory, electrical outlet receptacles, power cord receptacles, timer units, fuses, terminal blocks, transformers, or WIFI switches (not shown), wireless technology components such as WIFI/RF/BLUETOOTH transmitters and/or receivers). The UV light unit 100 may be mounted to objects via a mounting unit 126, illustrated herein as a bracket with an adjustable yoke or clamp via adjusting knob 128. The UV light unit 100 may also include one or more power connector 130 with bracket 132, held in place by bracket 132, to electrically connect two or more UV light units 100.

The UV light unit 100 may include a WIFI switch with power cord (not shown), such as a SONOFF WIFI connector, to control one or more of the above described functionalities, such as to turn ON/OFF the one or more sanitizing/disinfecting light sources 102 via an app. The clean mode/sanitize mode (such as how long the sanitizing/disinfecting light sources 102 are turned on) may also be controlled by an app. The UV light unit 100 may be configured with radiofrequency (RF) control or operation. The UV light unit 100 may utilize other wireless technology, such as BLUETOOTH.

The UV light unit 10 or the UV light unit 100 may be configured to be a smart device, and include the necessary hardware and/or software. As such, any one of the features of the UV light unit 10 or the UV light unit 100 may be controlled or operated remotely. The UV light unit 10 or the UV light unit 100 may be configured to communicate with an electronic device, such as a smart phone or computer tablet, i.e. IPAD, or remote Fab device. Control of the UV light/non-UV light, and/or air purification/cleaning functioning, i.e. on/off, scheduling of on/off times, UV/non UV light run times, when each of the one or more sanitizing/disinfecting light sources 102 turns on/off may also be accomplished using a cell phone or other remote electronic device. If needed, the UV light unit 10 or the UV light unit 100 may include a control panel (including a processor, memory, and other hardware or software) with display, operatively connected to one or more structures to drive functionality, allowing the user to turn on/off UV/non-UV lights, air purification/cleaning, turn off audio or visual alarms, or program on/off times or cycles by touch screen.

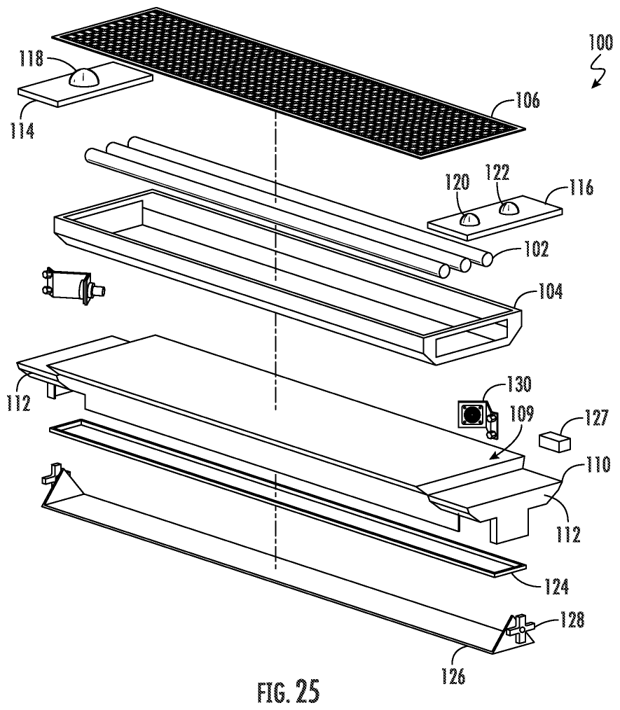

The UV light unit 100 (as well as UV light unit 10) may also optionally include one or more ionizer(s) 127 (see FIG. 25), such as the D5 Sterionizer manufactured by Eagle X Pro. Preferably, the ionizer 127 is designed to pump ions, such as 10 billion ions per second, without producing ozone. The positive and negative ions attach to microbes, such as virus/bacteria, breaks them down and renders them unable to infect. The ionizer 204 may be placed and secured to any portion of the UV light unit 100.

The UV light unit 100 may also be configured to provide air purification/cleaning. The UV light unit 100 (and the UV light unit 10) may be configured for UVC disinfection recirculation of air, at, for example, 280 cubic feet of air per minute. The UV light unit 100 may include an air filter accessory. The air filter accessory is preferably a modular add on device that is designed for simplified installation. The air filter accessory attaches to the back of the UV light unit 100 with screws. Intake and out vent plugs built within the unit are removed. A wire for power is attached via a quick connect plug. The air filter accessory module may be added to the web application and ran whenever required or on demand.

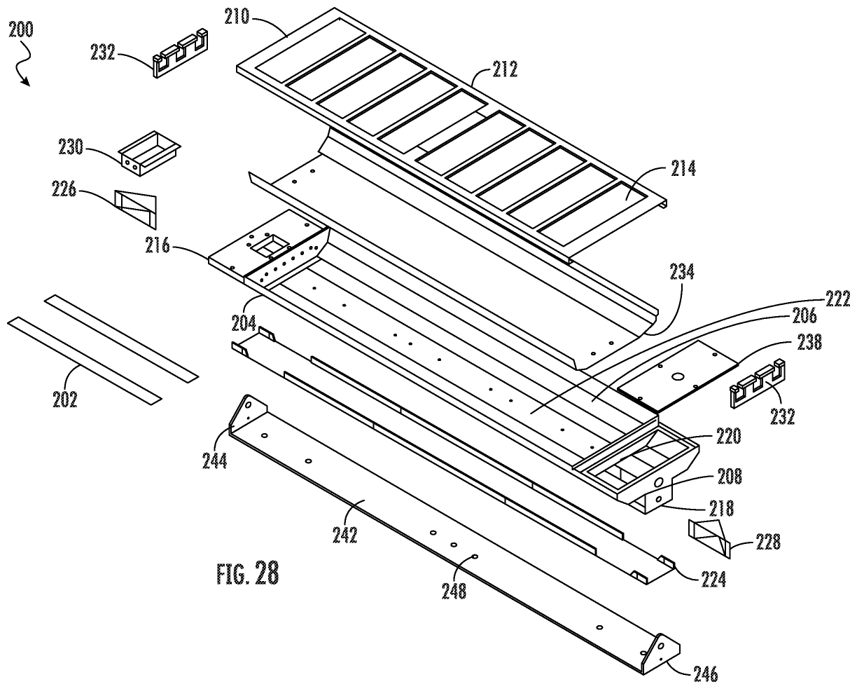

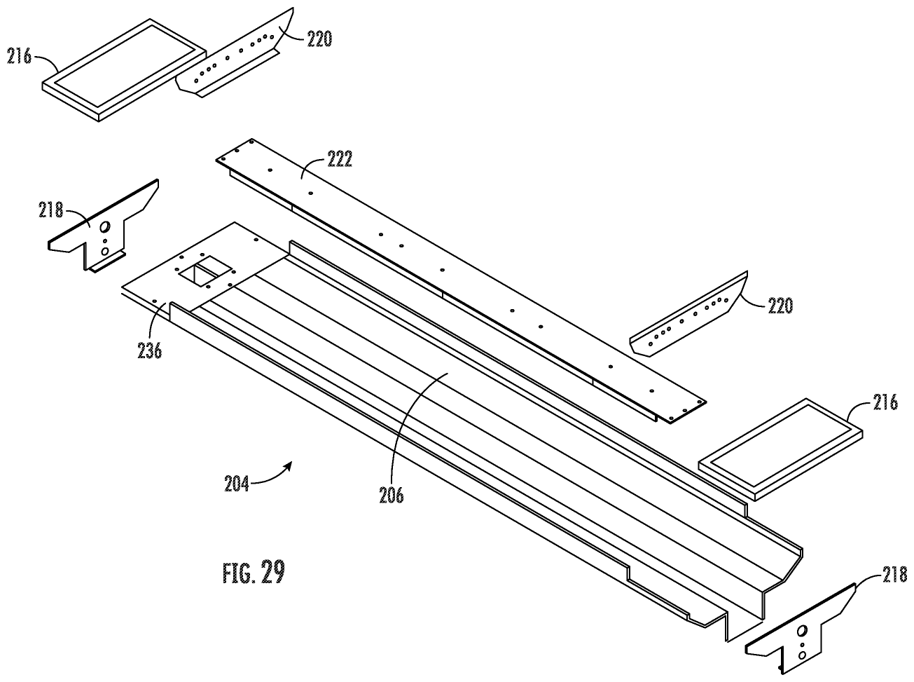

Referring to FIGS. 27-29, an alternative embodiment of the UV light unit, referred to generally as UV light unit 200, is shown. The UV light unit 200 illustrated in these figures includes the light source housing unit only. Accordingly, the UV light unit 200 may include any of the features or functional components as that described for the UV light unit 10 or UV light unit 100, where applicable. The UV light unit 200 is configured to expose surfaces, areas, and spaces to UV light sufficient to disinfect and/or sanitize the surfaces, areas, and spaces and 2) provide air filtration/purification or cleaning. The UV light unit 200 may be made of any suitable material, such as stainless steel, or combinations of suitable materials. Similar to the UV light unit 10 or 100, the UV light unit 200 may include one or more sanitizing/disinfecting light sources, such as a standard UV light lamp or an LED UV lamp (and optionally, non-UV lights). The UV light unit 200 may be configured so that when powered ON (OFF), all of the one or more sanitizing/disinfecting light sources turn ON (OFF) and emit light at the same time, or each individual sanitizing/disinfecting light source may be turned ON (OFF) and emit light separately and independently.

The one or more sanitizing/disinfecting light sources may be stored or placed within a light source frame assembly 204. The light source frame assembly 204 may include a pan side 206 and pan side emitter 208 and a light source cover 210. The light source cover 210 contains an elongated body 212 having louvers or a plurality of angled slats or strips 214 to allow air and light to pass through. The light source frame assembly 204 may include an end cap bracket 216, an outside end cap 218, and an inside end cap 220. The light source frame assembly 204 may further include a ballast cover 222 sized and shaped to cover a ballast (not shown) and a driver cover 224 sized and shaped to cover a driver (not shown). The light source frame assembly 204 is configured to hold or store various functional components and may include a plurality of brackets, such as a power bracket 226, an emitter bracket 228, and a sensor bracket 230, or a socket cover 232. To aid in light distribution, the light source frame assembly 204 may include a reflector 234). A plate sensor 236 may be placed at one end, with a plate alarm 238 placed at a second opposing end. The light source frame assembly 204 may be attached to a secondary structure, such as a stand, frame, or other light fixture, via an attachment member 240. The attachment member 240 is illustrated herein as a yoke having an elongated body with attachment ends or brackets 244 and 246. Openings 248 allow screws to be inserted, thus securing the light source frame assembly 204 to the secondary structure.

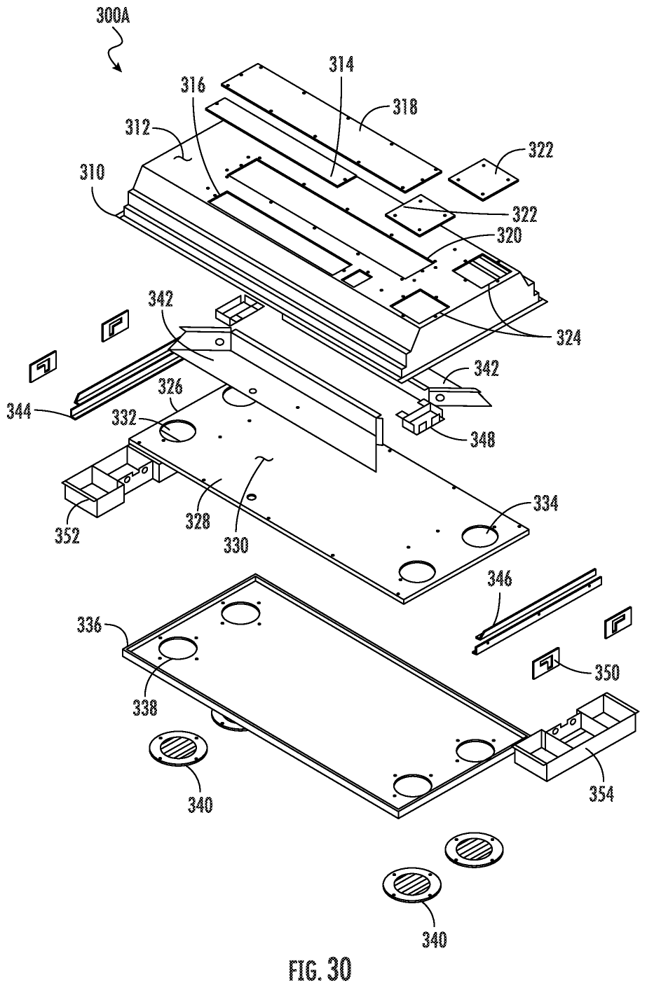

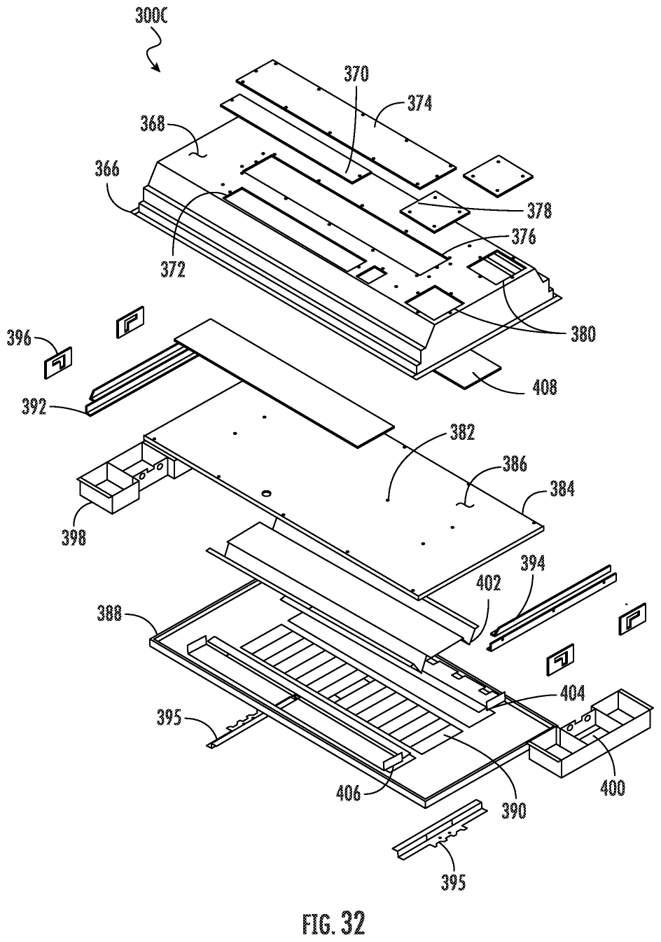

Referring to FIGS. 30-32, alternative embodiments of the UV light unit, referred to generally as UV light unit 300A, UV light unit 300B, or UV light unit 300C, are shown. The UV light units 300A, 300B, or 300C illustrated in these figures include the light source housing unit only. Accordingly, the UV light units 300A, 300B, or 300C may include any of the features or functional components (i.e. lights, fans, electrical components, sensors, safety features, wireless technology, etc.) as that described for the UV light unit 10 or UV light unit 100, where applicable. The UV light units 300A, 300B, or 300C are configured to expose surfaces, areas, and spaces to UV light sufficient to disinfect and/or sanitize the surfaces, areas, and spaces, and 2) provide air filtration/purification or cleaning. The UV light units 200, 300A, 300B and 300C may be made of any suitable material, such as stainless steel, or combinations of suitable materials. Similar to the UV light unit 10 or 100, the UV light units 300A, 300B, or 300C may include one or more sanitizing/disinfecting light sources, such as a standard UV light lamp or an LED UV lamp (and optionally, non-UV lights). The UV light units 300A, 300B, or 300C may be configured so that when powered ON (OFF), all of the one or more sanitizing/disinfecting light sources turn ON (OFF) and emit light at the same time, or each individual sanitizing/disinfecting light source may be turned ON (OFF) and emit light separately and independently.

The UV light unit 300A may comprise an upper frame or pan 310 having an upper surface 312 having an access cover 314 which covers opening 316 and a light source or radiator cover 318 covering opening 320 attached thereto. The upper frame or pan 310 may include fan access cover 322, which covers fan opening 324. An air plate 326 may be sized to extend the length of and/or fit within the upper frame or pan 310. The air plate 326 is shown as an elongated planar body 328 having a surface 330 allowing air to move there along (thus forming an air filtration/purification or cleaning chamber), while being exposed to the one or more sanitizing/disinfecting light sources. The air plate 326 may include air inlet openings 332 and opposing (on the opposite end) air outlet openings 334. An outer door or cover 336 may be removably secured to the upper frame or pan 310. The outer door or cover 336 may include a plurality of openings 338 with air covers 340, such as air louvers. The internal compartment of the UV light unit 300A may include a lower boot 342, support structures such as an upper bracket 344 and a lower bracket 346, socket bar 348, clips (hurricane clips) 350, and top seals 352 and 354.

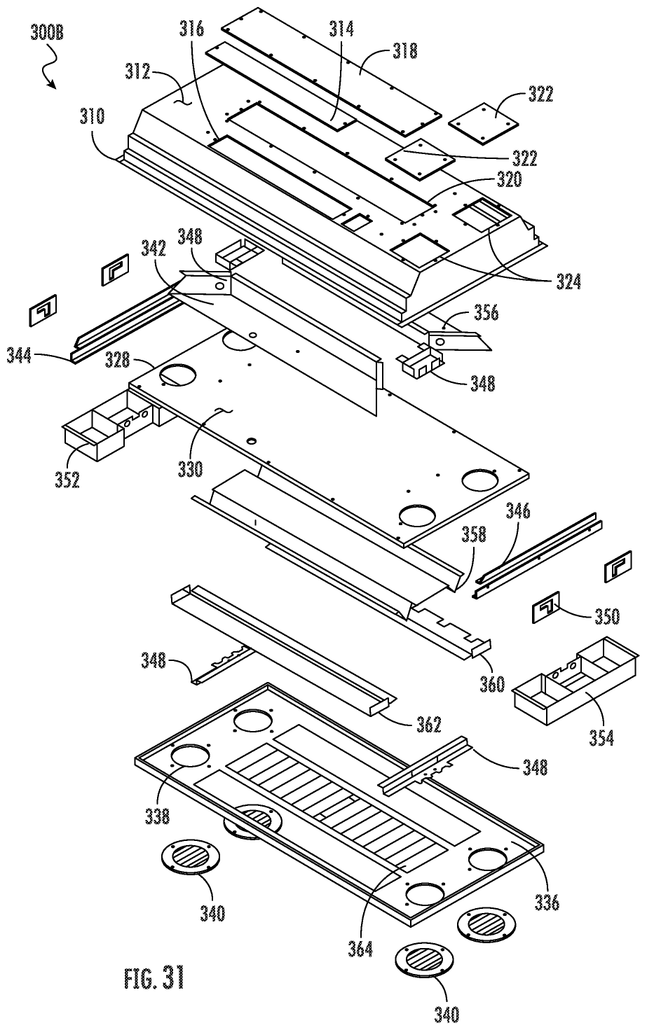

The light unit 300B, illustrated in FIG. 31, includes the same structures as described for light unit 300A (indicated by the same reference numbers reflecting the same structures as there described), along with several additional structural components. The light unit 300B may also include a lens 356 (UV light lens for UV lights or non-UV light lens for non-UV lights), a middle reflector 358, a drive cover 360, and a ballast cover 362. The outer door or cover 336 may include light louvers 364.

The UV light unit 300C may comprise an upper frame or pan 366 having an upper surface 368 having an access cover 370 which covers opening 372 and a light source or radiator cover 374 covering opening 376 attached thereto. The upper frame or pan 366 may include a fan access cover 378, which covers fan opening 380. An air plate 382 may be sized to extend the length of and/or fit within the upper frame or pan 366. The air plate 382 is shown as an elongated planar body 384 having a surface 386 allowing air to move there along (thus forming an air filtration/purification or cleaning chamber), while being exposed to the one or more sanitizing/disinfecting light sources. An outer door or cover 388 may be removably secured to the upper frame or pan 310. The outer door or cover 388 may include angled slots or light louvers 390. The internal compartment of the UV light unit 300C may include support structures, such as an upper bracket 392 and a lower bracket 394, socket bar or bracket 395, clips (hurricane clips) 396, and top seals 398 and 400. The UV light unit 300C may also include a reflector 402, a driver cover 404, a ballast cover 406 and a lens 408, (UV light lens for UV lights or non-UV light lens for non-UV lights).

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A light unit for exposing surfaces, areas, and spaces to light sufficient to disinfect and/or sanitize said surfaces, areas, and spaces comprising:
a housing unit configured to hold and operate one or more sanitizing/disinfecting light sources; and
an air filtration/purification chamber configured to expose air to said one or more sanitizing/disinfecting light sources.

2. The light unit for exposing surfaces, areas, and spaces to light sufficient to disinfect and/or sanitize said surfaces, areas, and spaces according to claim 1, wherein said one or more sanitizing/disinfecting light sources produce UV light.

3. The light unit for exposing surfaces, areas, and spaces to light sufficient to disinfect and/or sanitize said surfaces, areas, and spaces according to claim 2, wherein said one or more sanitizing/disinfecting light sources are LED UVC bulbs.

4. The light unit for exposing surfaces, areas, and spaces to light sufficient to disinfect and/or sanitize said surfaces, areas, and spaces according to claim 3, wherein said one or more sanitizing/disinfecting light sources are LED UVC bulbs emitting in the range of 200-400 nm.

5. The light unit for exposing surfaces, areas, and spaces to light sufficient to disinfect and/or sanitize said surfaces, areas, and spaces according to claim 1 further including one or more non-sanitizing/disinfecting light sources.

6. The light unit for exposing surfaces, areas, and spaces to light sufficient to disinfect and/or sanitize said surfaces, areas, and spaces according to claim 1 further including a motion sensor, wherein said one or more sanitizing/disinfecting light sources are configured to stop operating when motion is detected.

7. The light unit for exposing surfaces, areas, and spaces to light sufficient to disinfect and/or sanitize said surfaces, areas, and spaces according to claim 5 further including a motion sensor, wherein said one or more sanitizing/disinfecting light sources are configured to stop operating when motion is detected and said one or more non-sanitizing/disinfecting light sources are configured to operate.

8. The light unit for exposing surfaces, areas, and spaces to light sufficient to disinfect and/or sanitize said surfaces, areas, and spaces according to claim 1 further including one or more safety features that indicate that said one or more sanitizing/disinfecting light sources are operational.

9. The light unit for exposing surfaces, areas, and spaces to light sufficient to disinfect and/or sanitize said surfaces, areas, and spaces according to claim 8, wherein said one or more safety features that indicate that said one or more sanitizing/disinfecting light sources are operational is a light indicator.

10. The light unit for exposing surfaces, areas, and spaces to light sufficient to disinfect and/or sanitize said surfaces, areas, and spaces according to claim 8, wherein said one or more safety features that indicate that said one or more sanitizing/disinfecting light sources is an audible alarm indicator.

11. The light unit for exposing surfaces, areas, and spaces to light sufficient to disinfect and/or sanitize said surfaces, areas, and spaces according to claim 1, wherein light unit is configured to operate via wireless technology.

12. The light unit for exposing surfaces, areas, and spaces to light sufficient to disinfect and/or sanitize said surfaces, areas, and spaces according to claim 11, wherein said wireless technology is WiFi, RF, or Bluetooth.

13. The light unit for exposing surfaces, areas, and spaces to light sufficient to disinfect and/or sanitize said surfaces, areas, and spaces according to claim 1, wherein said light unit is configured to communicate with a smart phone or a computer tablet.

14. The light unit for exposing surfaces, areas, and spaces to light sufficient to disinfect and/or sanitize said surfaces, areas, and spaces according to claim 11, further including an ionizer.
